(19) United States
(12) Patent Application Publication
(10) Pub. No.: US 2023/0073902 A1
(43) Pub. Date: Mar. 9, 2023

(54) SYRINGE AND SYRINGE BODY

(71) Applicant: Fresenius Kabi Deutschland GmbH, Bad Homburg (DE)

(72) Inventors: Torsten Brandenburger, Reichelsheim (DE); Michael Schönhofen, Saarlouis (DE)

(21) Appl. No.: 17/759,772

(22) PCT Filed: Apr. 22, 2021
(86) PCT No.: PCT/EP2021/060468

§ 371 Date: Jul. 29, 2022

(30) Foreign Application Priority Data
Apr. 24, 2020 (EP) EP20171394

(51) Int. Cl. A61M 5/34; A61M 5/31

(52) U.S. Cl. CPC A61M 5/347; A61M 5/3137

(57) ABSTRACT

The invention relates to a syringe body having a nozzle and a threaded connection. The syringe body consists of an inner part and an outer part, the inner part comprising, at least in regions, a cycloolefin, in particular a cycloolefin copolymer or a cycloolefin polymer, as a first material. The first material forms an inner wall of the syringe body. The threaded connection of the outer part comprises, at least in regions, a material different from the cycloolefin as a second material. The invention further relates to a syringe having the syringe body.

The present application is a U.S. National Stage of PCT International Patent Application No. PCT/EP2021/060468, filed Apr. 22, 2021, which claims priority to EP Application No. 20171394, filed Apr. 24, 2020, both of which are hereby incorporated herein by reference.

### FIELD OF THE INVENTION

The invention relates to a syringe pre-filled with a medical fluid, comprising a syringe body having a Luer lock connection as well as to a method for manufacturing the syringe body.

### BACKGROUND OF THE INVENTION

Pre-filled syringes, which are also designated as single-use syringes, can comprise a syringe body of plastic. In order to deliver the medical fluid contained therein, the syringe body has a nozzle on its front side. The nozzle serves e.g. to connect a needle or a hose of a transfer system. Syringes with a male Luer lock connection are in particular widely used. In this case, the nozzle, which is formed conically as standard, is surrounded by a sleeve with an internal thread.

In particular in the case of pre-filled syringes, a suitable plastic material should meet different requirements.

The plastic material should be suitable for as many different medical fluids as possible. In particular a diffusion of components of the medical fluid into the material and leakage of material components through the medical fluid should largely be avoided, even when stored for a long time. Additionally, a suitable plastic material should also be autoclavable.

Cycloolefin copolymers have become particularly suitable materials. On the one hand, they can be easily processed in the injection-molding process. On the other hand, they are characterized by a high rigidity and high hardness whilst also being relatively low density. Such materials are also amorphous and highly transparent. They also have a low water absorption and a low water vapor permeability.

These plastics can, however, be relatively brittle. Cycloolefin copolymers have for example a relatively low elongation at break compared to polypropylene or polyethylene. This can for example lead to the Luer lock thread of the syringe being damaged when the connection of the transfer system is tightened improperly. Due to the brittleness of the material, the material cannot deform considerably plastically such that damage of this type can take place quite abruptly. Thus, the user has almost no tactile feedback that they are tightening the connection too much.

To reduce this problem, the published patent application DE 10 2017 112 823 A1 provides a strengthened portion of the threaded sleeve using ribs. The mechanical stability of the threaded connection is improved in this way.

The published patent application EP 3 342 441 A1 proposes to provide the threaded connection of a different material compared to the syringe body. It is in particular provided that the connection piece of weaker material is molded on the front side of the syringe main body.

### OBJECT OF THE INVENTION

Therefore, the object underlying the invention is to at least reduce the mentioned problems of the state of the art.

It is in particular an object of the invention to provide a syringe of plastic pre-filled with a medical fluid, which comprises an inner wall of a material with higher chemical resistance and good barrier effect, and which is also provided with a very stable threaded connection of plastic.

### SUMMARY OF THE INVENTION

The object of the invention is achieved by a syringe body according to claim 1 as well as a syringe having this syringe body.

Preferred embodiments and further developments of the invention can be inferred from the subject matter of the dependent claims, the description as well as the drawings.

The invention a syringe body having a nozzle and a threaded connection, with the syringe body comprising an inner part, which forms an inner wall of the syringe body and which comprises, at least in regions, a cycloolefin, in particular a cycloolefin copolymer or a cycloolefin polymer, as a first material, and with the threaded connection being formed as part of an outer part of a plastic, which comprises, at least in regions, a material different from the cycloolefin as a second material, with the outer part extending, at least in regions, around the side wall of the syringe body.

The invention is thus based on the fundamental idea that the syringe body is formed of an inner part and an outer part, with inner part and outer part being formed of different materials.

The invention in particular provides that at least the inner wall, preferably the entire inner wall of the syringe body is formed of a cycloolefin.

A cycloolefin within the meaning of the invention is understood as all cycloolefin copolymers, which are obtained by catalyzed copolymerization of cycloolefins.

Within the meaning of the invention, cycloolefins are also understood as materials, which are obtained by a ring-opening salt metathesis. These are, strictly speaking, not designated as cycloolefin copolymers, but as cycloolefin polymers.

The first material preferably consists at least 50% by weight, particularly preferably at least 90% by weight, of a cycloolefin.

Such materials are amorphous, transparent and, to a significantly lesser degree than other plastics, interact with the pre-filled medical fluid, in particular with an aqueous medical fluid.

In particular, this may concern so-called crystal clear polymers.

The outer part, which comprises the threaded connection, can, in contrast, consist of a different plastic, in particular a plastic, which is more mechanically stable.

Since the outer part extends around the side wall of the syringe body, torques are not transferred via the threaded connection to the inner part when a corresponding connection is screwed in.

The outer part serves as a grip in order to hold the tip when a connector is screwed into the threaded connection.

The user thus holds the syringe in particular at the side wall on the outer part, which also comprises the threaded connection. Thus, torques are not transferred to the inner part.

Inner part and outer part therefore tend to loosen less.

The inner part can be overmolded with the outer part.

A form-fitting connection, e.g. snapping on, is also provided according to another embodiment.

Inner part and outer part can also be welded or adhered together.

In the case of one embodiment, inner part and outer part are inserted into one another. Frictional forces between the walls are already sufficient to hold inner part and outer part together.

The outer part preferably extends around the entire side wall of the inner part.

The outer part can have recesses, in particular when it is formed opaquely.

The outer part is, however, preferably transparent.

In the case of one embodiment of the invention, a proximal grip of the syringe or of the syringe body is part of the outer part. In particular, only the outer part comprises a proximal grip extending radially outwardly.

Thus, a syringe can be easily provided, in which the grip is also formed of the mechanically more robust material.

The wall thickness of the inner part is, in the case of one embodiment of the invention, 0.3 to 3 times, preferably 0.5 to 1.5 times, the wall thickness of the adjoining wall of the outer part. This applies in particular to the side wall. In particular, inner part and outer part have roughly the same wall thickness.

The inner part thus has a relatively high wall thickness and therefore leads to a good barrier effect, in particular for water vapor and/or oxygen.

The total wall thickness, in particular in the region of the side wall, can e.g. be between 0.3 mm and 5 mm, preferably between 0.6 mm and 2.5 mm.

In the case of one embodiment of the invention, the outer part also extends around the nozzle of the inner part.

Thus, the female connection piece is slid over the outer part when the connector is connected. Axial forces acting on the inner part, which could loosen the inner part from the outer part, are thus avoided.

When filled as a finished syringe, a closure for the nozzle, for example a seal, preferably engages into the nozzle. Thus, the material of the outer part does not come into contact with the fluid in the internal volume of the syringe when the syringe is stored.

In the case of a further development of the invention, the inner part comprises a structuring on the front side of its end wall, into which a complementary structuring engages on the rear side of the outer part.

The syringe body comprises at least one two-layer region, inside of which first and second material comprise a structuring engaging into one another.

Due to the inner part and the outer part, the syringe body is, at least in regions, structured in a two-layered manner.

This embodiment of the invention is provided both for a syringe, in which inner part and outer part are connected together in a form-fitting manner, and for a syringe with outer part connected to the inner part in a materially-bonded manner.

A structuring is understood as a regular or irregular vertical profile of the inner part with protrusions or depressions. The second material of the outer part, which is molded for example in an injection-molding process, engages into the depressions.

The areas of the first material and the second material adjoining one another at a contact area or boundary area inside the two-layer region thus each comprise a surface relief with a profile structure which is at least partially filled by the respectively other material.

It has been found that the tendency of the two layers to lead to a separation of the materials, in particular to separate during autoclaving and/or during mechanical strains, can be notably reduced.

Stresses, for instance due to different thermal expansion coefficients of the materials, are distributed, owing to peaks and valleys, not only substantially radially along a flat surface, but are partially deflected in the axial direction.

The linear thermal expansion coefficient for cycloolefin copolymers (under standard conditions) is usually below 60×10⁻⁶×K⁻¹. Polypropylenes, in contrast, have for example a higher thermal longitudinal expansion coefficient, in particular of over 150×10⁻⁶×K⁻¹.

In spite of the strength and a certain brittleness of the cycloolefin copolymer, in particular in the case of an autoclaving process due to different thermal expansion of both materials, a breakage of the materially-bonded connection does not occur.

The size of the surface of a boundary area between the first and the second material is also enlarged by the structuring. This boundary area is preferably larger by at least 20% compared to an unstructured configuration.

Lastly, a form-fitting connection between the first and the second material can be provided by the structuring, in particular with respect to a torque between the parts, when a connector is connected to the threaded connection.

An improved mechanical connection is hereby provided in a very simple manner.

The size of the surface of a boundary area or contact area between the first and the second material is also enlarged by the structuring. This boundary or contact area is preferably larger by at least 20% compared to an unstructured configuration.

Lastly, a form-fitting connection between the first and the second material can be provided by the structuring, in particular with respect to a torque between the inner part and the outer part.

An improved mechanical connection is hereby provided in a very simple manner.

The manufacture of such a syringe is also not associated with significantly increased effort. Thus, only an injection molding tool, which comprises a negative profile in the form of the profiling of the inner part, needs to be used to manufacture the inner part.

When structuring is mentioned below, this can be the structuring of the inner part and also the structuring of the outer part, in particular on its end walls.

These structurings preferably have a complementary configuration.

The protrusions can be formed as regular and/or irregular protrusions. The depressions can also be formed as regular and/or irregular depressions.

In the case of one embodiment of the invention, the protrusions and/or the depressions are formed as concentric rings. In the case of such profiling, the protrusions and the depressions run in particular radially from inside outwards. As a result, there is in particular a uniform deflection of stresses in the axial direction all around.

The protrusions or depressions can in particular have a substantially triangular cross-section. In the case of this embodiment, the profiling is formed as a saw tooth profile. However, other structures, such as for example a wave shape, in particular a sinusoidal shape, nubs engaging into one another, etc., are also possible.

In the case of one embodiment of the invention, elements of the structuring of the first material are formed conically. The elements of the structuring, e.g. the webs or nubs, taper upwards. This facilitates provision of the structuring of the second layer by injection molding the second material. In particular, the tendency for bubble formation in the region adjoining the structural elements is reduced.

In the case of one embodiment of the invention, the structuring comprises on the front side of the end wall of the inner part at least one web and/or one groove, which extends from the nozzle, at least in regions, in the direction of the side wall. Thus, a torsion-proof connection is, so to speak, provided. The elements of the structuring can also be formed as nubs and/or wedges.

In one embodiment, the structuring of the end wall of the inner part can have at least one, preferably a plurality of, webs and/or grooves extending radially. The webs or grooves can hereby have any desired cross-section. However, they are preferably formed with a saw tooth profile.

Owing to the radial alignment, web and groove each form a form-fitting connection, which acts as a torsion-proof connection.

In particular, the structuring in the top view can have a fan-like and/or star-like configuration. For example, the rods of a fan are formed by radially extending webs and the covering is formed between the webs by the profile extending in a ring-shaped manner. The fan extends around the threaded connection and thereby occupies 360° of the end surface.

The structuring can have between 3 and 16, preferably between 6 and 10 radially extending webs and/or grooves distributed around the circumference.

The form-fitting connection provided by the structuring serves in particular to improve the strength of the connection during torque stress, such as for example when tightly screwing in the connection.

In the case of a further development of the invention, the outer part of the second material is formed thickened around the threaded connection compared to a radially adjoining region of the outer part or the end wall of the outer part of the second material.

Thus, the material of the second layer is reinforced by a thickened portion, such as for example a bead or an inner bevel, in the transition region from the threaded connection to the end wall. The danger of the threaded connection breaking off when tightened too much is hereby reduced.

In particular, when using a soft second material, the deformation of the material in the region of the end wall is also reduced during tightening, whereby the tendency of the materially-bonded connection to loosen in this reduced is in turn reduced.

The second material is preferably a plastic, in particular a transparent plastic, which can be processed in the injection-molding process.

The second material preferably has a higher notch impact toughness than the first material. The second material in particular has a notch impact toughness at least 20%, preferably at least 50%, higher than the first material.

The notch impact toughness is a material characteristic value which defines the tendency of the material to form cracks during dynamic strain. This is determined in the notch bend impact test. The dynamic bending due to the sudden strain causes a break, often without the flow of the material observed during slow strain.

In the context of the invention, all material characteristic values, i.e. in particular the notch impact toughness and the material characteristic values, such as elasticity modulus and hardness, are determined for standard conditions, i.e. 20° C. and 50% humidity.

The notch impact toughness in the context of the invention is determined according to DIN ISO 179-1 (11/2010).

In this case, a test body with a notch standardized in its shape is strained by an impact pendulum. The impact pendulum notches the test body or penetrates it with a defined kinetic energy. Then, the notch is measured or, in the case of penetration, the height at which the pendulum swings back is maintained. From the weight of the impact pendulum and the difference of the pendulum start and end position, the impact energy used can be calculated, with the impact energy representing the product of sample cross-section and notch impact toughness.

Cycloolefins generally have a notch impact toughness of under 3 kJ/m². In contrast, the second material should have a higher notch impact toughness, in particular of over 3.5 kJ/m², preferably of over 5 kJ/m².

A polypropylene can in particular be used as the second material.

In particular, partially-crystallized polypropylene has good mechanical properties and is also transparent.

The second material can in particular be softer than the first material.

In particular, the second material can have a Shore hardness D (according to ISO 7619-1 (2/2012) of below 75, in particular of below 70.

The cycloolefin, in contrast, has a Shore hardness of over 80 in one embodiment of the invention.

In the case of one embodiment of the invention, the second material has a lower elasticity modulus than the first material.

In particular, the elasticity modulus of the second material (according to DIN ISO 527-1 (2/2019) is between 1000 and 1800 MPa. The elasticity modulus of the cycloolefin can in particular be between 1800 and 2200 MPa.

The elongation at break is in particular also an important parameter for the second material.

According to one embodiment of the invention, the second material has an elongation at break at least 1.5 times, preferably at least 5 times and particularly preferably at least 10 times, higher than the first material.

The elongation at break is also determined according to DIN ISO 527-1. It is indicated as a %.

The cycloolefins used can e.g. have an elongation at break of below 5%, whereas for example a polypropylene used can have an elongation at break of 100% or more.

In order to break the threaded connection, the material must be deformed beyond the upper yield limit. The subsequent deformation in the plastic region of a material with high elongation at break is generally noticed by a trained user such that they recognize that the load-bearing limit of the connection is now actually almost exceeded.

In one embodiment of the invention, the structuring on the front side of the end wall of the inner part is in particular delimited by a radially circumferential web. The structuring ends inside the end wall of the inner part.

The web can in particular be formed by a front section of the side wall of the inner part.

According to one embodiment, the structuring of the first and/or of the second material has a maximum structure depth of over 0.1 mm, preferably of over 0.25 mm, and/or of less than 1 mm, preferably less than 0.5 mm. The maximum structure depth is understood as the vertical difference between the tip of a protrusion and the bottom of a depression. The structuring is thus a structuring in the macro level order of magnitude, whereby force deflection described above is effectively achieved.

The distance from protrusion to protrusion or depression to depression, the so-called structure width, is, in one embodiment of the invention, more than 0.2 mm, preferably more than 0.5 mm, and/or less than 3 mm, preferably less than 1.5 mm.

According to one embodiment of the invention, the ratio of maximum structure depth to structure width can also be more than 0.1, preferably more than 0.25 and/or below 1, preferably below 0.5.

The wall thickness of the layer of the first material is, in the region of the end wall in one embodiment of the invention, 0.3 to 3 times, preferably 0.5 to 1.5 times, the wall thickness of the layer of the second material. The total wall thickness, in particular in the region of the end wall formed in a two-layered manner at least in regions, can e.g. be between 0.3 mm and 5 mm, preferably between 0.6 mm and 2.5 mm.

A syringe comprising a syringe body according to one of the embodiments described above is within the field of the invention, with the syringe having a plug with which the nozzle is sealed, a plunger and also in particular a plunger rod for delivering the medical fluid via the nozzle. The plunger rod can already be pre-mounted on the plunger or be provided separately.

The syringe according to the invention is preferably filled with a medical fluid, in particular with a fluid containing a medication.

The syringe is a pre-filled syringe in this exemplary embodiment.

In particular, the syringe is located in a preferably oxygen-impermeable outer packaging, for example a tear-open foil packaging. The syringe, preferably the packaged syringe, is in particular autoclaved.

In particular, the syringe is located in a preferably oxygen-impermeable outer packaging, for example a tear-open foil packaging.

The syringe has been autoclaved in this outer packaging, for example at a temperature of over 110° C., preferably over 120° C. and is thus completely sterile.

According to one embodiment, the medical fluid is an oxygen-sensitive medicinal fluid, for example a medicinal emulsion. According to one embodiment, the medicinal fluid is or comprises propofol, in particular a propofol emulsion. Propofol is described by the chemical name 2,6-Diisopropylphenol (IUAPC).

The syringe body can for example be manufactured using an injection-molding process.

In this case, an inner part of a cycloolefin is first molded as the first material.

To this end, an injection-molding tool is used, which comprises the negative of a structuring of the end wall of the inner part such that an inner part with a structured end wall is created.

Then, an outer part with a threaded connection of a second material is molded around the inner part.

The outer part hereby automatically forms a complementary structure in the region of the structured end wall of the syringe body, which, as mentioned above, significantly improves the mechanical connection of both materials.

### BRIEF DESCRIPTION OF THE DRAWINGS

The subject matter of the invention will be explained in more detail below on the basis of exemplary embodiments in accordance with FIG. 1 to FIG. 10.

FIG. 1 is a side view of an exemplary embodiment of a syringe according to the invention.

FIG. 2 is an axial sectioned view along the line A-A of FIG. 1.

FIG. 3 is an axial sectioned view of the syringe body.

FIG. 4 shows an alternative exemplary embodiment of a syringe body.

FIG. 5 is a detail view of the region D of FIG. 3.

FIG. 6 is a perspective view of the head piece of the syringe.

FIG. 7 is a perspective view of the head piece of the inner part.

FIG. 8 is a top view of the end wall of the syringe body.

FIG. 9 is a sectioned view along the line B according to FIG. 8.

FIG. 10 is a sectioned view along the line C according to FIG. 8.

### DETAILED DESCRIPTION OF THE DRAWINGS

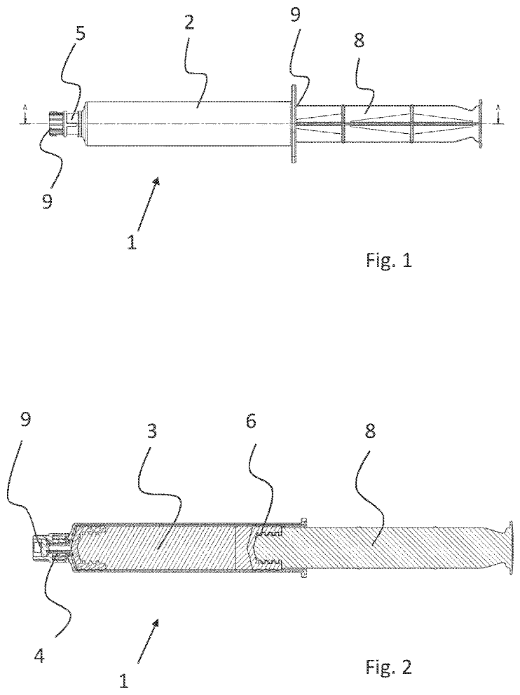

FIG. 1 shows, in a side view, an exemplary embodiment of a syringe 1 according to the invention.

The syringe 1 comprises a syringe body 2 having a threaded connection 5 and a proximal grip 9.

As represented in the axial sectioned view according to FIG. 2, the syringe body 2 provides an inner volume 3 which is pre-filled with a medical fluid. A gaseous volume in the inner volume 3 compensates for pressure fluctuations and in particular prevents the syringe body 2 from deforming during pressure fluctuations.

The nozzle 4 is sealed with a seal 9, which preferably engages into the nozzle 4 such that the outer wall of the nozzle 4 does not come into contact with the medical fluid at least in the sealed state.

Nozzle 4 and threaded connection 5 form the connection of the syringe 1, which is formed in particular as the male Luer lock connection.

The medical fluid can be delivered through the nozzle 4 via the plunger rod 8 connected to the plunger 6.

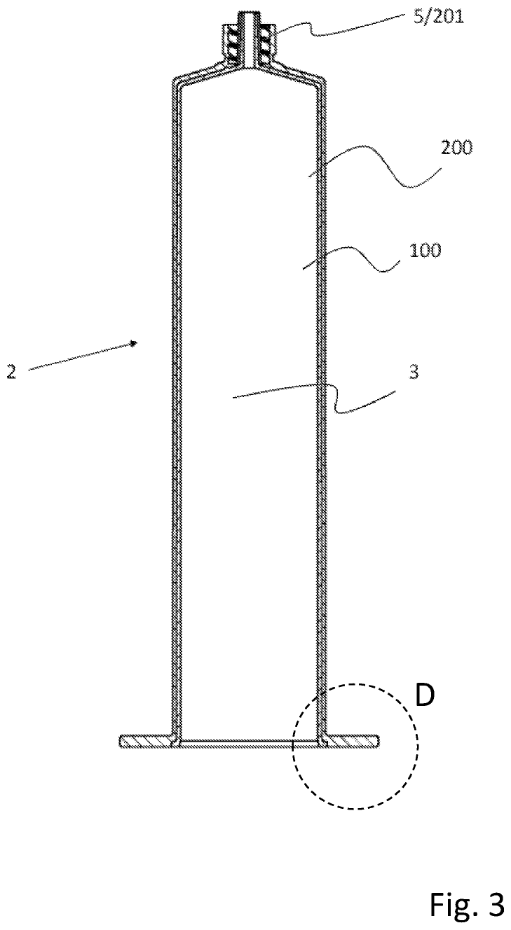

FIG. 3 shows, in an axial sectioned view, only the syringe body 2.

The syringe body 2 is formed in two parts and comprises an inner part 100 of cycloolefin as well as an outer part 200, which is formed of a mechanically more stable material than the inner part 100, in particular of a polypropylene.

The threaded connection 5/201 is provided only by the outer part 200.

The invention is provided for syringes with almost any desired volume, in particular 1 ml to 100 ml.

In the exemplary embodiment according to FIG. 3, a relatively large syringe, in particular with an inner volume 3 of roughly 50 ml, is represented.

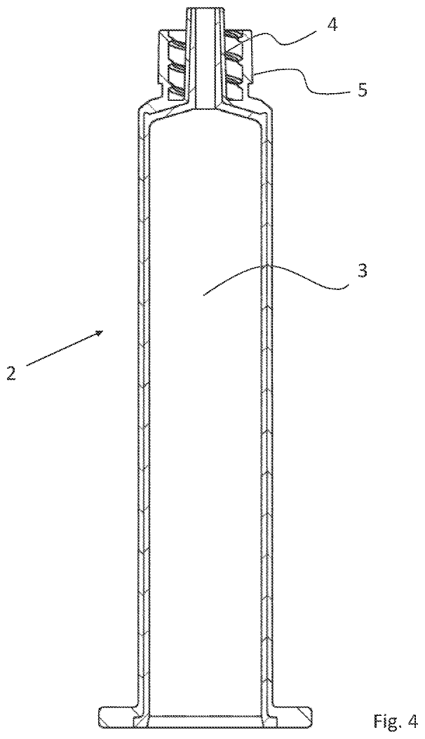

FIG. 4 is an axial sectioned view of an alternative embodiment of a syringe body 2.

This exemplary embodiment concerns a relatively small syringe, in particular with an inner volume 3 of roughly 5 ml. The basic structure of the syringe body 2, however, remains unchanged.

In this case, the dimensions of the connection, consisting of nozzle 4 and threaded connection 5, do not differ.

Consequently, the threaded connection 5 has a significantly larger diameter in proportion to the maximum diameter of the syringe body 2.

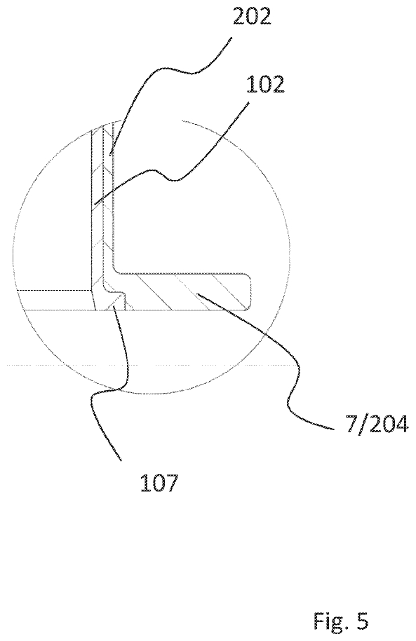

FIG. 5 is a detail representation of the region D of FIG. 3, i.e. of the proximal end of the syringe body 2.

The side wall of the syringe body 2 is formed in a two-layered manner and is formed of the side wall 102 of the inner part and of the adjoining side wall 202 of the outer part 200.

The grip 7/204, in contrast, consists, at least in regions, only of the material of the outer part 200.

In this embodiment, the inner part 100 has a proximal collar 107, which protrudes into the grip 204 and e.g. the axial positioning in one embodiment, in which inner part 100 and outer part 200 are pushed into one another, has improved or which, in one embodiment where the inner part 100 is overmolded with the outer part 200, makes the grip 6 more rigid.

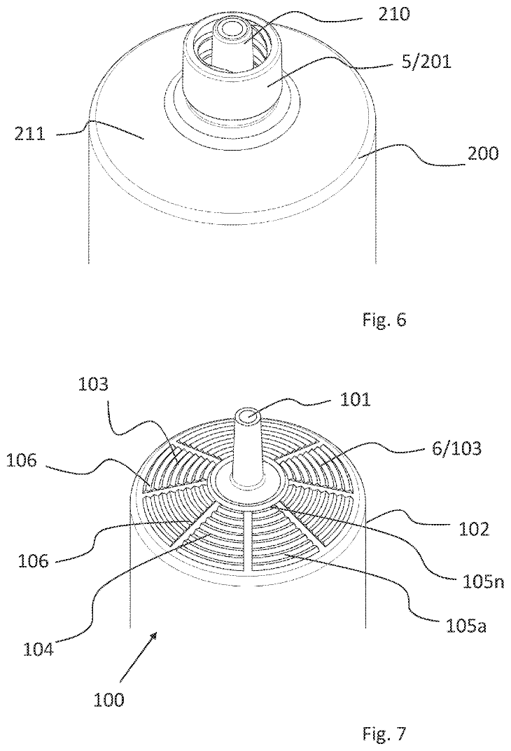

FIG. 6 shows, in a perspective view, the head piece of a syringe 1 according to one exemplary embodiment of the invention.

The syringe body 2 is, as mentioned above, formed of inner part 100 and outer part 200, such that, in this view from the inner part 100, at most the inner wall of the nozzle 4 can be discerned.

The threaded connection 5/201 with an internal thread extends around the nozzle 4/210, which is formed in a two-layered manner.

FIG. 7 shows, in a corresponding perspective view, only the inner part 100 of cycloolefin.

The end wall 103 of the inner part 100 comprises a structuring 104, which has preferably been provided by injection-molding.

In this exemplary embodiment, the structuring 104 of the end wall 103 comprises a plurality of rings 105a-105n extending concentrically around the nozzle 101 of the inner part 100, which are formed as elevations or protrusions.

These rings 105a-105n are interrupted by a plurality of radially extending grooves 106, which form a torsion-proof connection in interaction with the outer part 200.

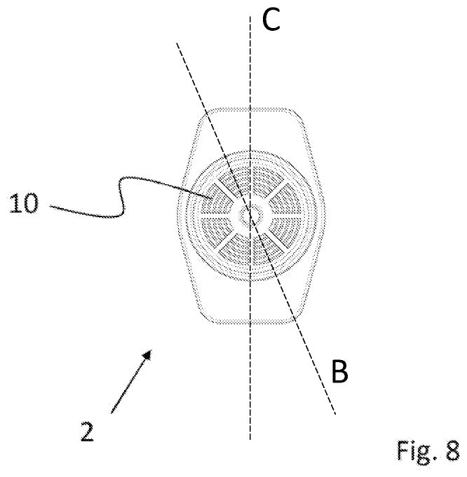

FIG. 8 is a top view of the distal side of the syringe body 2.

The end wall of the syringe body 2 comprises a region 7 formed in a two-layered manner, in which the structurings 104, 208 of inner part 100 and outer part 200 engages into one another.

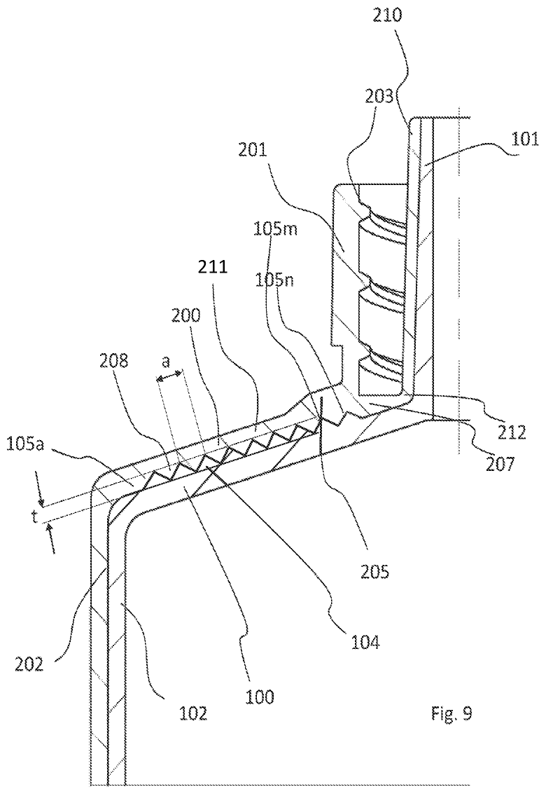

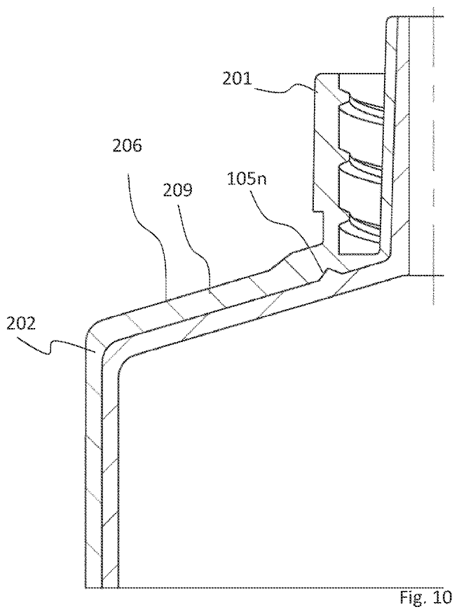

The structuring engaging into one another formed according to the invention is thus, as in particular shown in the detail representation according to FIG. 9 along the section line B according to FIG. 8, formed by the structuring 104 of the inner part 100 as well as by the structuring 208 of the end wall 211 of the outer part 200 engaging into this structuring 104.

The structuring 104 of the inner part 100 comprises a plurality of teeth 105a-105n in a radial sectioned view. The structuring 104 is thus formed, in this exemplary embodiment, in an axial section as a saw tooth profile.

The correspondingly formed teeth of the second material of the outer part 200 engage between the teeth 105a-105n.

The second material extends around the outer walls of the entire inner part 100.

The structuring (104/208) is, in this exemplary embodiment, formed as a regular structuring, in which the distance from peak to peak or valley to valley (or protrusion to protrusion or depression to depression), i.e. the structure width, is defined by the distance a between two tips or two valleys.

The structuring 104/208 also has a maximum depth t, which defines the structure depth, and which defines the vertical distance from the peak tip to valley bottom.

The threaded connection 201 with the teeth 203 of the internal thread, which is located opposite the nozzle 210, merges via a bead 205 in the region of the second material into the end wall 211 of the outer part 200.

The mechanical strength is hereby increased in this region such that the risk of the threaded connection 201 breaking is reduced.

Inside the threaded connection 201, the bottom 207 of the threaded connection 201 reaches up to an inner corner 212 at which the end wall 211 of the outer part 200 merges into the nozzle 210.

The nozzle 4 is thus formed in a two-layered manner and namely of the nozzle 101 of the inner part 100, which provides the inner wall of the nozzle 4, and of the nozzle 210 of the outer part 200, which provides the outer wall of the nozzle 4.

FIG. 10 is a sectioned view along the line C of FIG. 8.

The section now runs through a radial groove 106 of the inner part 100. The groove 106 is formed such that the teeth or rings 105a-105m of the structuring 104 are interrupted in a radially extending strip.

A corresponding web 209 of the second material is formed in the groove 106.

This web forms a torsion-proof connection in interaction with the groove 106.

In this exemplary embodiment, a ring 105n reaching directly to the threaded connection 201 is not recessed by the groove 106.

In the region of the web 209, this ring also forms a deflection point via which the input of stresses into the region of the web 209 is reduced.

Inner part 100 and outer part 200 can be connected in a materially-bonded manner, in particular by the inner part 100 being overmolded with the outer part 200. Alternatively, it is possible to connect inner part 100 and outer part 200 to one another by welding or adhering or by a form-fitting and/or force-fitting connection.

Through the invention, a syringe 1 can be provided which is more stable, in particular less prone to brittle fracture and has improved haptics. The required quantity of cycloolefin can also be reduced.

### LIST OF REFERENCE NUMERALS

1 Syringe
2 Syringe body
3 Inner volume
4 Nozzle
5 Threaded connection
6 Plunger
7 Grip
8 Plunger rod
9 Seal
10 Two-layer region with structuring
100 Inner part
101 Nozzle
102 Side wall
103 End wall
104 Structuring of the inner part
105a-105n Ring/tooth
106 Radial groove
107 Collar
200 Outer part
201 Threaded connection
202 Side wall
203 Tooth
204 Grip
205 Bead
206 Upper side
207 Bottom
208 Structuring of the outer part
209 Web
210 Nozzle
211 End wall
212 Corner

1. A syringe body for a medical syringe, comprising:
a nozzle, a threaded connection, and an inner part, which forms an inner wall of the syringe body and which comprises, at least in regions, a cycloolefin as a first material, the inner part including a side wall,
wherein the threaded connection is formed as part of an outer part of a plastic, which comprises, at least in regions, a material different from the cycloolefin as a second material,
wherein the outer part extends, at least in regions, around the side wall of the inner part.

2. The syringe body according to claim 1, wherein the outer part extends around the entire side wall of the inner part and/or in that a grip of the syringe body is part of the outer part.

3. The syringe body according to claim 1, wherein the inner part and the outer part are inserted into one another and, at least in regions, are connected in a materially-bonded manner.

4. The syringe body syringe according to claim 1, wherein the inner part is overmolded with the outer part.

5. The syringe body according to claim 1, wherein a wall thickness of the inner part in the region of the side wall and/or an end wall is 0.3 to 3 times, a wall thickness of an adjoining wall of the outer part.

6. The syringe body according to claim 1, wherein a front side of an end wall of the inner part comprises a structuring into which a complementary structuring of the outer part engages.

7. The syringe body according to claim 6, wherein the structuring of the inner part and/or the complementary structuring of the outer part is in each case formed as a profile with a plurality of protrusions and/or depressions.

8. The syringe body according to claim 6, wherein the structuring of the inner part and/or the complementary structuring of the outer part has at least one radially extending groove or a radially extending web.

9. The syringe body according to claim 1, wherein a layer of the second material is formed thickened around the threaded connection compared to a radially adjoining region of the layer of the second material.

10. The syringe body according to claim 1, wherein the second material has a higher notch impact toughness than the first material.

11. The syringe body according to claim 1, wherein the second material is softer than the first material.

12. The syringe body according to claim 6, wherein the structuring on the front side of the end wall of the inner part is delimited by a radially circumferential web.

13. A syringe comprising a syringe body according to claim 1.

14. The syringe according to claim 13, wherein the nozzle is sealed with a seal preferably engaging into the nozzle and further comprising a plunger with a plunger rod for delivering a medical fluid through the nozzle.

15. The syringe according to claim 13, wherein the syringe is arranged in an outer packaging impermeable to oxygen.

16. The syringe body according to claim 5, wherein the wall thickness of the inner part in the region of the side wall and/or the end wall is 0.5 to 1.5 times the wall thickness of the adjoining wall of the outer part.

17. The syringe body according to claim 8, wherein the structuring of the inner part and/or the complementary structuring of the outer part has a plurality of radially extending webs and/or grooves.

18. The syringe body according to claim 10, wherein the second material has a notch impact toughness of at least 20% higher than the first material.

19. The syringe body according to claim 18, wherein the second material has a notch impact toughness of at least 50% higher than the first material.
